(19) United States
(12) Patent Application Publication
(10) Pub. No.: US 2024/0023811 A1
(43) Pub. Date: Jan. 25, 2024

(54) Systems for Biomarker Detection and Methods Thereof

(71) Applicant: California Institute of Technology, Pasadena, CA (US)

(72) Inventors: Shouleh Nikzad, Valencia, CA (US); Samuel R. Cheng, Walnut, CA (US)

(73) Assignee: California Institute of Technology, Pasadena, CA (US)

(21) Appl. No.: 18/356,027

(22) Filed: Jul. 20, 2023

Related U.S. Application Data
(60) Provisional application No. 63/390,704, filed on Jul. 20, 2022.

(51) Int. Cl. A61B 5/00; A61B 1/04; A61B 1/06; A61B 1/00

(52) U.S. Cl. CPC A61B 5/0071; A61B 5/0077; A61B 1/043; A61B 1/0684; A61B 1/00032; A61B 1/00016; A61B 1/00048

(57) ABSTRACT

Systems and method for detecting light emitting biomarkers are described. The detection systems can be tuned to detect desired wavelengths emitted from biomarkers. The compact and cost-effective detection systems can provide detection results of the biomarkers in a timely manner.

### CROSS-REFERENCE TO RELATED APPLICATIONS

The current application claims the benefit of and priority under U.S.C. § 119(e) to U.S. Provisional Patent Application No. 63/390,704 entitled “Query Probe (QProbe)” filed Jul. 20, 2022. The disclosure of U.S. Provisional Patent Application No. 63/390,704 is incorporated herein by reference in its entirety for all purposes.

### GOVERNMENT SPONSORED RESEARCH

The invention was made with U.S. Government support under Grant No. 80NM00018D0004 awarded by NASA (JPL). The government has certain rights in the invention.

### FIELD OF THE INVENTION

The present invention generally relates to diagnostic systems; and more particularly to diagnostic systems using photodiodes to detect light emitting biomarkers.

### BACKGROUND OF THE INVENTION

Photodiodes are light-sensitive semiconductor diodes. When activated by light, a photodiode can absorb photons and create a current. The current can be measured to determine characteristics of the activating light. A photodiode can be activated by light with a specific wavelength range. Ultraviolet (UV) light has a shorter wavelength than visible light. Background visible light can interfere with the detection of UV light.

When activated by light of a specific wavelength, certain biomarkers and bacteria reflect UV light. Biomarkers can also be labeled to emit a specific UV wavelength that can later be detected.

### SUMMARY OF THE INVENTION

Many embodiments are directed to systems of biomarker detection system devices and associated methods thereof.

One embodiment of the invention includes a biomarker detection system comprising: at least one light emitting diode, wherein the at least one light emitting diode emits an excitation light that excites a biomarker such that the biomarker auto-fluoresces and emits a light in a wavelength from 100 nm to 450 nm; at least one light detecting device positioned adjacent the at least one light emitting diode, wherein the at least one light detecting device detects the emitted light and generates a signal; and at least one signal processor, wherein the at least one signal processor processes the signal to generate a detection result of the biomarker.

In another embodiment, the at least one light detecting device comprises a photodiode, a photodiode array, a photodetector, a photodetector array, a CMOS camera, a CCD camera, or a combination thereof.

In an additional embodiment, the at least one light emitting diode is on a curved substrate.

In a further embodiment, the at least one light detecting device comprises an anti-reflective coating that is selectively transmissible to the emitted light of the biomarker.

Another embodiment further comprises a power source selected from the group consisting of: an AC power supply, a DC power supply, a battery, and a combination thereof.

An additional embodiment further comprises a handle, wherein the power source and the at least one signal processor are housed in the handle.

A further yet embodiment comprises a signal transmitter selected from the group consisting of: a USB cable, a Bluetooth transmitter, a wireless transmitter, and a combination thereof, wherein the signal transmitter transmits the detection result to a at least one display.

In another further embodiment, the at least one display is a part of portable device selected from the group consisting of a phone, a tablet, and a laptop computer.

In yet another embodiment, the detection system is configured to insert into a body cavity.

In a yet further embodiment, the detection system is configured to survey a surface area.

In a further embodiment again, the at least one detection device is positioned on top of the at least one light emitting diode, wherein the at least one detection device is transmissible to the excitation light.

In yet another embodiment, the at least one detection device and the at least one light emitting diode are positioned in alternating layers.

Another embodiment includes a biomarker detection system comprising: at least one optical element on a convex surface of a curved substrate; at least one light emitting diode positioned on a circumference of the at least one optical element, wherein the at least one light emitting diode emits an excitation light that excites a biomarker such that the biomarker auto-fluoresces and emits a light in a wavelength from 100 nm to 450 nm; at least one light detecting device positioned on a concave surface of the curved substrate, wherein the at least one optical element focuses the emitted light to the at least one light detecting device and the at least one light detecting device detects the emitted light and generates a signal; and at least one signal processor, wherein the at least one signal processor processes the signal to generate a detection result of the biomarker.

In an additional embodiment, the at least one light detecting device comprises a photodiode, a photodiode array, a photodetector, a photodetector array, a CMOS camera, a CCD camera, or a combination thereof.

In yet another embodiment, the at least one light detecting device comprises an anti-reflective coating that is selectively transmissible to the emitted light.

Another further embodiment comprises a power source selected from the group consisting of: an AC power supply, a DC power supply, a battery, and a combination thereof.

A further yet embodiment comprises a handle, wherein the power source and the at least one signal processor are housed in the handle.

Another yet embodiment further comprises a signal transmitter selected from the group consisting of: a USB cable, a Bluetooth transmitter, a wireless transmitter, and a combination thereof, wherein the signal transmitter transmits the detection result to a at least one display.

In another embodiment again, the at least one display is a part of portable device selected from the group consisting of a phone, a tablet, and a laptop computer.

In a further embodiment again, the detection system is configured to insert into a body cavity.

In another further embodiment, the detection system is configured to survey a surface area.

An additional embodiment includes a method for detecting biomarkers comprising:
exciting at least one biomarker with at least one light emitting device, wherein the biomarker auto-fluoresces and emits a light in a wavelength from 100 nm to 450 nm;
capturing the emitted light with at least one light detecting device;
processing a signal from the emitted light; and
outputting a detection result of the at least one biomarker.

In a further embodiment again, the at least one light emitting device comprises a light emitting diode, a laser, and any combination thereof.

In yet another embodiment, the at least one light detecting device comprises a photodiode, a photodiode array, a photodetector, a photodetector array, a CMOS camera, a CCD camera, and any combination thereof.

In a further embodiment, the at least one light detecting device comprises an anti-reflective coating that is selectively transmissible to the emitted light of the biomarker.

In an additional embodiment again, the at least one biomarker is in a body cavity.

In yet another embodiment, the at least one biomarker in on a surface.

Additional embodiments and features are set forth in part in the description that follows, and in part will become apparent to those skilled in the art upon examination of the specification or may be learned by the practice of the disclosure. A further understanding of the nature and advantages of the present disclosure may be realized by reference to the remaining portions of the specification and the drawings, which forms a part of this disclosure.

### BRIEF DESCRIPTION OF THE DRAWINGS

FIG. 1 illustrates a process for detecting biomarkers in accordance with an embodiment.

FIGS. 2A and 2B conceptually illustrate detection system probes in accordance with an embodiment.

FIG. 3A conceptually illustrates a detection system with cameras in accordance with an embodiment.

FIGS. 3B and 3C conceptually illustrate interior views of a detection system camera in accordance with an embodiment.

### DETAILED DISCLOSURE OF THE INVENTION

Many embodiments implement compact and portable detection systems for detecting light emitted from biomarkers. Detection systems in accordance with various embodiments can be tuned to detect a plurality of wavelengths. In several embodiments, detection systems can detect autofluorescence of biomarkers in ultraviolet (UV) wavelength ranges and/or outside UV wavelength ranges. Various auto-fluorescent bacteria and/or molecules can emit light when they absorb light without added fluorescent markers and/or probes. Detection systems can detect autofluorescence of biomarkers such as bacteria and/or molecules without labeling the biomarkers. Examples of autofluorescence biomarkers can include bacteria such as (but not limited to) Escherichia colt, Salmonella Typhimurium, Staphylococcus aureus, and/or molecules such as (but not limited to) chlorophyll, collagen, retinol, riboflavin, NAD(P)H, folic acid, pyridoxine, tyrosine, dityrosine, and/or flavin. As can be readily appreciated, any of a variety of autofluorescence species or molecules can be detected using detection systems as appropriate to the requirements of specific applications in accordance with various embodiments of the invention.

In several embodiments, detection systems can incorporate light emitting devices such as (but not limited to) light emitting diodes (LEDs) and/or lasers; light detecting devices such as (but not limited to) photodiodes, photodiode arrays, photodetectors, photodetector arrays, CMOS cameras, and/or CCD cameras; power supplies such as (but not limited to) AC power supplies, DC power supplies, and/or batteries; signal transmitters such as (but not limited to) USB cables and/or wireless Bluetooth transmitters. Light emitting devices in accordance with some embodiments can provide excitation wavelengths for the target biomarkers. Excitation wavelengths for various biomarkers can vary from about 250 nm to about 700 nm; or from about 250 nm to about 300 nm; or from about 250 nm to about 350 nm; or from about 250 nm to about 400 nm; or from about 250 nm to about 450 nm; or from about 250 nm to about 500 nm; or from about 250 nm to about 550 nm; or from about 250 nm to about 600 nm; or from about 250 nm to about 650 nm. As can be readily appreciated, any of a variety of excitation wavelength can be used as appropriate to the requirements of specific applications in accordance with various embodiments of the invention. In some embodiments, light emitting devices can have at least one dimension ranging from about 50 nm to about 1 mm.

In various embodiments, light detecting devices can be imaging sensors that detect photons and generate electrical signals. Imaging sensors such as photodiodes and/or photodiode arrays can be formed on flexible substrates and/or curved substrates such as concave or convex substrates. Flexible and/or curved substrates enable sensors to conform to any shapes or geometries that might be needed for detecting auto-fluorescent biomarkers. In certain embodiments, light detecting devices can be illuminated on the front side (front-illuminated) and/or on the back side (back-illuminated). Light detecting devices in accordance with some embodiments can be formed independently and affixed to a substrate of the biomarker detection system. In a number of embodiments, light detecting devices can be formed together with the light emitting devices and then affixed to a substrate of the biomarker detection system. In certain embodiments, light emitting devices can be positioned below the light detecting devices. In several embodiments, light detecting devices and light emitting devices can be formed as alternating layers in the biomarker detection systems. In some embodiments, light emitting devices can surround light detecting devices in the biomarker detection systems.

In order to achieve stable, high quantum efficiency (QE) in back-illuminated photodiodes (for example, silicon detectors), the surface of photodiodes can be passivated via delta-doping. (See, e.g., S. Nikzad, et al., “High Efficiency UV/Optical/NIR Detectors for Large Aperture Telescopes and UV Explorer Missions”, Journal of Astronomical Telescopes, Instruments, and Systems, Vol. 3, Issue 3, 036002 (2017); the disclosure of which is herein incorporated by reference.) The photosensitive volume of most silicon detectors comprises a thin layer of high purity epitaxial silicon that is grown on a thick, highly conductive silicon substrate. Highly conductive silicon makes a poor detector, as photogenerated electrons are lost to recombination before they can be detected. In order to achieve high QE in a back illuminated detector, the thick substrate can be removed to expose the thin layer of high purity silicon. Thinning (i.e., the substrate removal process) a silicon substrate that is hundreds of microns thick while leaving intact (about 5 to 15 μm thick) silicon detector can be achieved using wafer-level thinning processes. In addition, delta doping and superlattice doping processes can be used for surface passivation for light detecting devices in order to achieve stable QE and reduce surface generated dark current. Delta doping and superlattice doping are two-dimensional (2D) doping techniques achieved by low temperature molecular beam epitaxy (MBE). The use of low temperature MBE allows for control of the surface doping profile and band structure engineering with nearly atomic-scale precision. 2D doping enables low temperature crystalline growth of silicon with higher dopant concentrations, while achieving nearly 100% activation and high crystalline quality. Atomic boron deposited by MBE on an atomically clean silicon surface can form a self-organized, 2D phase with surface densities up to 0.5 monolayer (approximately 3.4×10¹⁴ B/cm² on a <100>silicon surface). Subsequent growth of epitaxial silicon encapsulates and stabilizes this 2D layer of boron in the silicon lattice. The process is termed “delta doping” because the resulting dopant profile resembles the mathematical delta function. “Superlattice doping” refers to a doping profile in which more than one delta layer is incorporated into the MBE structure.

Anti-reflective (AR) coatings can be applied to light detecting devices in order to improve detection accuracy and sensitivity. (See, e.g., J. Hennessy, et al., “Advances in detector-integrated filter coatings for the far ultraviolet”, Proceedings Volume 11821, UV, X-Ray, and Gamma-Ray Space Instrumentation for Astronomy XXII; 118211A (2021), the disclosure of which is herein incorporated by reference.) AR coatings such as materials and/or structures can be selected to filter light of specific wavelengths. In some embodiments, AR coatings can be applied to enhance detection of ultraviolet wavelengths for imaging sensors (CCD image sensors and/or CMOS image sensors). AR coatings can include various metal dielectric filter structures and/or graded thickness coatings. Such systems can achieve high quantum efficiency for the selected wavelengths. AR coatings on image sensors can detect wavelengths less than about 200 nm; or from about 100 nm to about 200 nm; or from about 100 nm to about 250 nm; or from about 100 nm to about 400 nm. Examples of AR coatings include metal and/or dielectric materials such as (but not limited to) silicon, silicon oxide, silicon nitride, aluminum, aluminum oxide, hafnium oxide, metal fluorides (such as magnesium fluoride, aluminum fluoride), and any combinations thereof. In many embodiments, AR coatings can include a plurality of layers of materials. Such layers can be flat layers (for example, a single layer has the same thickness throughout), graded layers (for example, a single layer has different thickness throughout), and a combination thereof. AR coatings can be deposited using a variety of techniques including (but not limited to) atomic layer deposition (ALD).

In many embodiments, biomarker detection systems can be compact and lightweight, and can provide quick and cost-efficient preliminary health diagnostic and/or screening. Biomarker detection systems may enable diagnostics to be performed in remote areas (for example, via telemedicine). Detection systems can include at least one UV enhanced light detecting device. Such detection systems can achieve high sensitivity to detect faint auto-fluorescence signals from biomarkers such as (but not limited to) bacteria, viruses, and/or molecules. Detection systems consume low power and are compact in size. The portable detection systems may allow for fast diagnosis that can query a high number of samples in a number of different environments such as (but not limited to) an airport or school. Detection systems can be affixed to probes of various sizes and/or geometries such that the probes can be used for detection of different samples. Some embodiments insert the detection probes into the body cavities such as (but not limited to) mouths, nasal canals, ear canals, and/or anal canals, of human targets and/or animal targets. Several embodiments use the detection probes to sample surfaces such as (but not limited to) tables, desks, beds, floors, handles, and/or floors, and detect possible biomarkers on the surfaces. The detection systems can achieve real time diagnosis by detection, identification, and quantification of biomarkers.

Portable detection systems can be used in special environments such as in space and/or remote areas with limited resources. The detection systems can operate accurately in such environments, and generate results in a timely manner with reduced cost compared to traditional detection tools. In addition, the detection systems can perform non-invasive imaging and detection via small entry points, on surfaces, and/or in cavities.

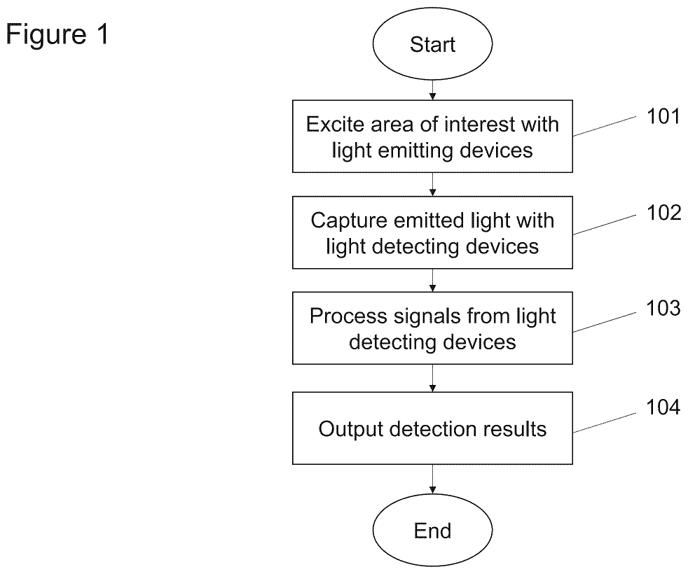

FIG. 1 illustrates a process for detecting biomarkers using the detection system in accordance with an embodiment. The detection systems can be placed to the desired location for biomarker detection. Excite (101) the area of interest using light emitting devices. The area of interest may contain autofluorescence biomarkers. The biomarkers can absorb the excitation light. The excited biomarkers emit light in response to the light emitting devices. Capture (102) the emitted light from the excited biomarkers using light detecting devices. The light detecting devices absorb the emitted photons and produce measurable signals such as current, voltage, and/or generate images. The light detecting devices can be tuned to be selective to the wavelength ranges of biomarker autofluorescence wavelengths. Light filters such as AR coatings can be applied to the light detecting devices to filter in desired wavelengths of light. Process (103) the measurable signals from the light detecting devices. The measurable signals can be related to biomarker species and/or concentrations. Output (104) the detection results based on the processed signals. The results can be presented in a form of a report, a statement, a text, or a description. The results can be sent to portable devices via a signal transmitter. The portable devices may comprise a processing system such as (but not limited to) smartphones, smart watches, tablets, laptop computers, or any combinations thereof.

In many embodiments, the detection systems can detect biomarkers in a body cavity. In various embodiments, the detection systems can be in a form of a detection probe (also referred as a query probe). In several embodiments, the detection system probe can be inserted into the body cavity of a patient to detect biomarkers. In some embodiments, the detection system comprises a probe housing and/or probe, a handle, a power source, a signal transmitter, at least one light detecting device, and at least one light emitting device. In some embodiments, the probe housing comprises a probe lined with light detecting devices and light emitting devices. The user can excite the biomarkers on the area of interest such as (but not limited to) surfaces or body cavities using the light emitting devices. The light detecting devices can capture the autofluorescence signals from the excited biomarkers. In some embodiments, the light detecting devices comprise UV sensitive light detectors such as (but not limited to) superlattice-doped and AR coated silicon detectors. The superlattice-doped detectors have increased sensitivity of detecting UV wavelengths. AR coating can filter out undesired wavelengths and allow desired wavelengths to pass through. In many embodiments the light detecting devices comprise photodiodes. The photodiodes can form on curved surfaces. The curved light detecting devices have the flexibility to adapt to different probe sizes. Small probes can reach into small crevices and/or body cavities. Large probes can be used for wide surface query.

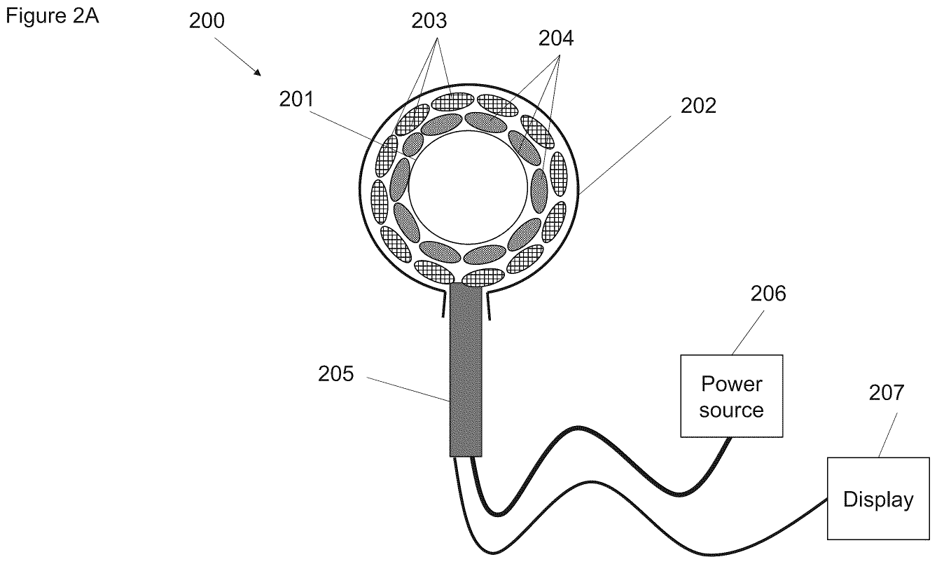

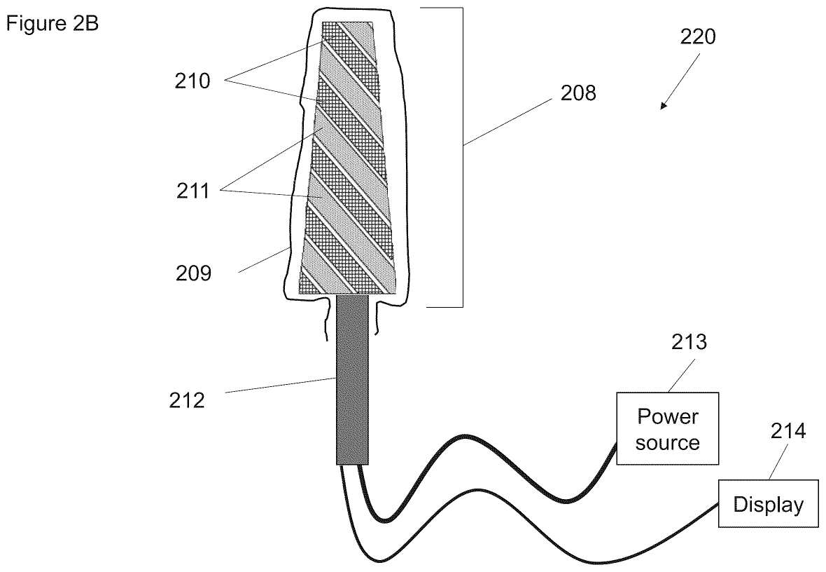

FIGS. 2A and 2B conceptually illustrate configurations of a detection probe in accordance with an embodiment. FIG. 2A conceptually illustrates cross section of a spherical detection probe 200. The detection probe 200 includes a housing 201. The detection probe housing 201 can be inserted into a subject's body cavity. The detection probe housing 201 may be encased in a transmittable sleeve 202 so the detection probe housing 201 can be used more than one time. The transmittable sleeve 202 can be modified to select desired wavelengths. The transmittable sleeve 202 can be tailored to transmit specific wavelengths including (but not limited to) less than or equal to about 450 nm; or from about 100 nm to about 200 nm; or from about 125 nm to about 175 nm; or from about 175 nm to about 225 nm; or from about 200 nm to about 300 nm; or from about 225 nm to about 275 nm; or from about 275 nm to about 325 nm; or from about 300 nm to about 400 nm; or from about 325 nm to about 375 nm; or from about 375 nm to about 425 nm. The transmittable sleeve 202 can be made of biocompatible materials that are suitable for detection in body cavity. After each detection, the transmittable sleeve 202 can be cleaned and reused. The cleaning step may not be necessary if the transmittable sleeve 202 can stay clean and functional and/or have self-cleaning capabilities.

Light detecting devices 203 such as (but not limited to) photodiodes and light emitting devices 204 such as (but not limited to) light emitting diodes (LEDs) can be formed on the surface of the detection probe housing 201. The light detecting devices 204 can be formed on curved substrates 201 (concave or convex substrates) such that they can be affixed to the surface of the spherical detection probe housing. As can be appreciated, although the probe housing 201 is illustrated in a spherical shape in FIG. 2A, any of a suitable shape can be used for the probe. Examples of probe shapes can include (but are not limited to) spheres, hemi-spheres, cones, ellipses, balloon shapes, dome shapes, cylindrical shapes, circular shapes, and any combinations thereof. Probes with rounded corners can be preferred as they may be less invasive when inserted in to body cavity. The light emitting devices 204 and the light detecting devices 203 may cover the whole circumference of the probe housing. The light emitting devices 204 and the light detecting devices 203 may cover less than or equal to about a quarter of the circumference of the probe housing; or less than or equal to about half of the circumference of the probe housing; or less than or equal to about three quarters of the circumference of the probe housing; or greater than about three quarters of the circumference of the probe housing. The light detecting devices 203 can be back-illuminated such that the light emitting devices 204 can be positioned on the back side of the light detecting devices 203. In various embodiments, the light emitting devices 204 (such as an array of light emitting devices) form a single layer underneath a single of the light detecting devices 203 (such as an array of light detecting devices), as shown in FIG. 2A. The light emitting devices 203 are transmissible to the excitation wavelengths such that the excitation light can travel through the light detecting devices 203 and excite biomarkers. The biomarkers absorb the excitation light and auto-fluoresce. The light detecting devices 203 detect the light emitted by the biomarkers. In many embodiments, the front side (the side that is facing the transmittable sleeve 202) of the light detecting devices 203 may have a metalized front layer (not shown) to insulate the light detecting devices 203 from noise signals. The light detecting devices 203 such as photodiodes can be modified with AR coatings to enhance light detection specificity and sensitivity. In several embodiments, the insulated and/or AR coated back-illuminated light detecting devices 203 can be deposited directly on the detection probe housing 201 substrate that is coated (wholly or partially) with light emitting devices 204. The light emitting devices 204 such as LEDs emit excitation wavelengths through the back-illuminated detection devices 203 to areas of interest comprising biomarkers.

The light detecting devices 203 and/or the light emitting devices 204 can vary in sizes from about 10 nanometers to about 1 cm. The sizes of the devices can be selected to adapt to the detection probe. The devices can be fabricated using CMOS compatible processes and technologies.

The detection probe 200 may comprise a handle 205 for user maneuverability in accordance with an embodiment. The detection probe 200 may be powered via an external power source 206 connected by a wire. The handle 205 may contain batteries to power the detection probe 200 such that an external power source 206 may not be necessary. Signals detected from the light detecting devices 203 can be processed and transmitted to a display 207 to output detection results. The detection probe 200 may comprise signal processers, signal transmitters, storage memory, in order to process the detected signals to output detection results. The detection results can be shown on a display 207. The display 207 can be a portion of a device such as (but not limited to) a computer, a portable device, a smartphone, a tablet, a laptop computer, or a smart watch.

FIG. 2B conceptually illustrates a detection probe 220 with a cylindrical probe housing 208. The detection housing 208 can be inserted into the body cavity of a patient. A removable transmittable sleeve 209 may encase the detection probe housing 208 such that the detection probe housing 208 can be used for more than one detection successively. The detection probe housing 208 may have a cylindrical shape. The cylindrical probe housing 208 may have rounded corners to reduce and/or prevent damage to body cavity. One end of the probe housing 208 can taper to a smaller diameter than the opposite end such that the probe can be inserted into small body cavity. Although the cylindrical probe has a tapered shape as shown in FIG. 2B, it can be readily appreciated that the probe can have an elongated shape such as (but not limited to) a cylinder, a cone, a triangle, an ellipse, a spiral, and any combinations thereof. A plurality of light detecting devices 210 and light emitting devices 211 can be deposited in alternating layers on the probe housing 208. The alternating layers can form a spiral. The light detecting devices 210 such as (but not limited to) photodiodes can be front-illuminated or back-illuminated. The light emitting devices 211 such as (but not limited to) LEDs can be placed on either or both sides of the light detecting devices 210. The light emitting devices 211 and the light detecting devices 210 can cover the cylindrical probe entirely or partially. The light detecting devices 210 can be modified with AR coatings to improve detection accuracy. The light emitting devices 211 can excite biomarkers with excitation wavelengths. The light detecting devices 210 can detect the emitted light of the biomarkers.

The detection probe 220 may comprise a handle 212 for user maneuverability in accordance with an embodiment. The detection probe 220 may be powered via an external power source 213 connected by a wire. The handle 212 may contain batteries to power the detection probe 220 such that an external power source 213 may not be necessary. Signals detected from the light detecting devices 210 can be processed and transmitted to a display 214 to output detection results. The detection probe 220 may comprise signal processers, signal transmitters, storage memory, in order to process the detected signals to output detection results. The detection results can be shown on a display 214. The display 214 can be a portion of a device such as (but not limited to) a computer, a portable device, a smartphone, a tablet, a laptop computer, or a smart watch.

In many embodiments, the detection system may comprise imaging devices such as (but not limited to) cameras for biomarker detection. In several embodiments the detection system with cameras can be inserted into the body cavity of a subject to detect biomarkers and/or diagnose a subject. The detection systems can be referred as DiaCam. In many embodiments, compact detection systems with cameras are capable of performing preliminary health diagnostics by detecting biomarkers (such as bacteria and/or molecules), identifying the biomarkers, and measuring the biomarker concentrations. Such diagnostic methods are cost effective and make health diagnostics more affordable and readily available. Compact and high-performance detection systems can be used in space exploration and/or on Earth.

The detection systems in accordance with some embodiments can accurately detect biomarkers by using cameras that are selectively sensitive to UV wavelengths (from about 100 nm to about 400 nm). In several embodiments, the detection systems with cameras can be lightweight, compact in size, and handheld. Such detection systems can provide quick and cost-efficient preliminary health diagnostics, and allow users to make informed decisions for seeking help from medical professionals. The low power consumption and high sensitivity of the detection systems allows for early detection and fast diagnosis that can query a high number of users in settings such as (but not limited to) offices or train stations. In many embodiments, the compactness of the detection systems allows diagnostics to be performed in remote areas (such as rural areas or regions in conflict) that may have difficult access to medical professionals.

In many embodiments, detection systems incorporate low power and compact UV enhanced CMOS cameras. The detection systems with cameras can be fabricated in different forms and can be integrated into various optical imaging instruments for biomarker detection. The detection systems with cameras can be handheld detection devices. In several embodiments, the detection systems with cameras can image surfaces to detect the presence of biomarkers and/or biomarker concentrations (such as bacteria and/or molecule levels. The surfaces can be a part of (but not limited to) tables, countertops, furniture, walls, and/or office items. In some embodiments, the detection systems with cameras can have a lollipop form and the detection systems can be inserted and/or fitted into body cavities, such as (but not limited to) mouth, nasal cavities, ear cavities, and/or anal cavities. Detection of the bacteria and/or molecule levels on the surface and/or cavities can provide information to alert the user to visit a medical professional.

In many embodiments, the detection systems comprise lens, light emitting devices, light detector housing, light detecting devices. In several embodiments the detection systems can communicate with handheld displays via wired connections or wirelessly. In some embodiments the handheld displays can have processing capabilities and are a part of a device such as (but not limited to) a smartphone, a tablet, a smart watch, and/or a laptop computer. In several embodiments, the light detecting devices can incorporate superlattice-doped silicon detectors for enhanced UV sensitivity. In many embodiments the light detecting devices can be arranged in an array and/or formed on a curved substrate. The curved light detecting devices have may allow the compactness of the detection systems. The curved light detecting devices and/or arrays enable the detection devices to be formed in different sizes. Small scale detection systems can reach into small crevices or body cavities and large-scale detection systems can be used for surface query. The curved light detecting devices may eliminate additional optical elements such as (but not limited to) field flatteners to reduce sizes and masses of the detection systems. The detection systems in accordance with embodiments can have increased throughput, image quality, and/or field of view with minimal optical elements such that autofluorescence from bacteria and/or molecules can be detected and quantified.

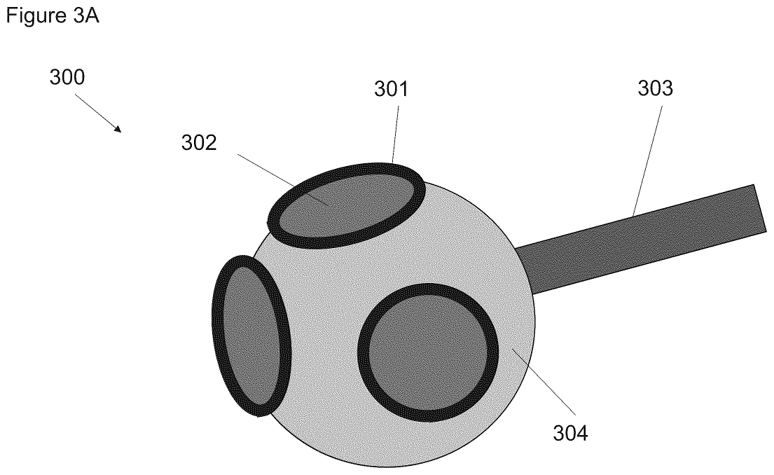

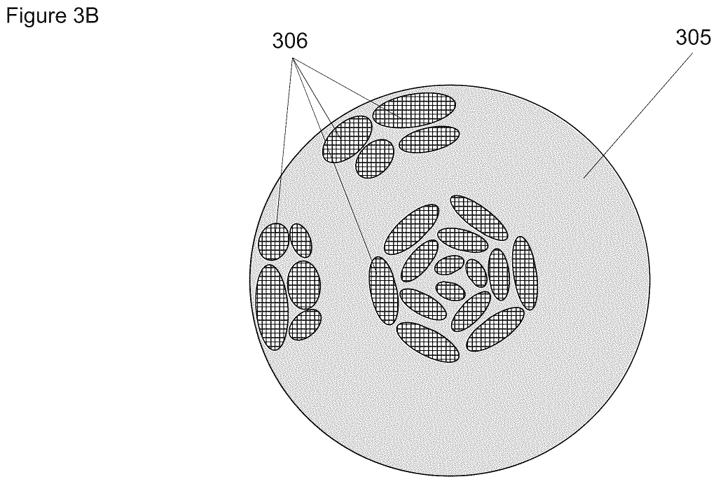

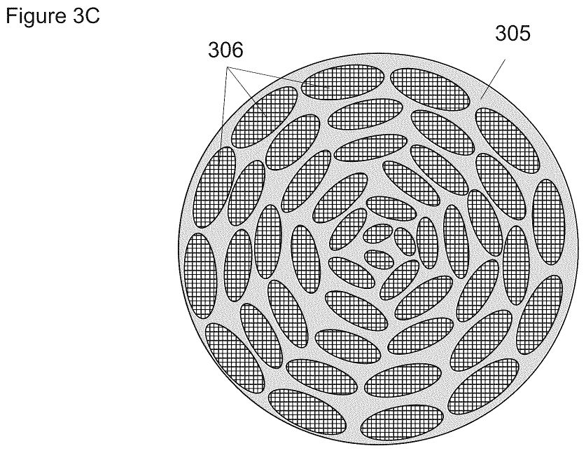

FIGS. 3A and 3B conceptually illustrate detection systems incorporating cameras in accordance with an embodiment. FIG. 3A conceptually illustrates a side view of the detection system in accordance with an embodiment. The detection system 300 can detect biomarkers on surfaces and/or body cavities. The detection system housing 304 encloses light detecting devices 306. The detection system housing 304 can have a spherical shape. As can be appreciated, although the housing 304 is illustrated in a spherical shape in FIG. 3A, any of a suitable shape can be used for the housing. Examples of detection system housing shapes can include (but are not limited to) spheres, hemi-spheres, cones, ellipses, cylinders, balloon shapes, dome shapes, cylindrical shapes, circular shapes, and any combinations thereof. The detection system comprises optical elements such as lenses 302. Light emitting devices 301 such as LEDs can surround the lenses 302. The light emitting devices 301 can excite biomarkers present on the area of interest. The light emitted from the excited biomarkers transmit through the lenses 302. Light detecting devices 306 such as photodiodes and/or photodiode arrays can be positioned underneath the lenses 302 to detect the light transmitted through the lenses 302. The light detecting devices 306 may be affixed to the interior surface 305 of the detection system housing 304. The lenses 302 may filter the transmitted light such that desired wavelengths are absorbed by the light detecting devices 306. The light detecting devices 306 such as photodiodes can be modified with AR coatings to enhance light detection specificity and sensitivity. The light detecting devices 306 can be formed on curved substrates to adapt to curved housing for the detection systems. The lenses 302 and the light detecting devices 306 can cover wholly or partially of the sphere surface. The light detecting devices 302 can be affixed to the optical element 302 surfaces facing the interior of the detection system housing 304. The light detecting devices 306 can be front-illuminated and/or back-illuminated. The light emitting devices 301 such as LEDs emit excitation wavelengths to areas of interest comprising biomarkers. The biomarkers absorb the excitation light and auto-fluoresce. The light detecting devices 306 detect the light emitted by the biomarkers.

The user can maneuver the detection system 300 to survey an area of interest using the handle 303. The light signal data detected by the light detecting devices 306 may be processed to output detection result. The processed results can be transmitted wirelessly to a display or at least one portable device. In some embodiments the portable device comprises processing capabilities, such as (but not limited to) a smartphone, a tablet, a laptop computer, and any combination thereof. The portable device outputs the transmitted light signal data results to the user. The wireless transmitter can be housed in the handle 303. The signal can be transmitted via a wired connection (not shown). The detection system 300 can be powered via a wired power source (not shown) or a wireless power source (such as a battery) housed in the handle 303.

FIG. 3B conceptually illustrates the interior surface of the detection system housing 305 in accordance with an embodiment. Light detecting devices 306 such as photodiodes and/or photodiode arrays can be formed as a plurality of arrays. The plurality of arrays can be affixed to the interior surface of the detection system housing 305. Each array of the light detecting devices 306 can be positioned underneath the lenses.

FIG. 3C conceptually illustrates the interior surface of the detection system housing 305 in accordance with an embodiment. Light detecting devices 306 such as photodiodes and/or photodiode arrays can be fabricated on a curved surface. The curved surface can form the interior surface of the detection system housing 305.

As can be readily appreciated, detection systems may include photodiodes of various geometries and configurations and/or AR coatings to detect desired wavelength ranges. In several embodiments, the AR coatings may be formed in a plurality of layers using different materials. The plurality of layers of AR coatings can have varied thicknesses and arrangements. In various embodiments the optical elements can be varied to allow specific wavelengths to enter the interior of the detection system camera housing such that only light emitted by the biomarkers may be detected by the light detecting devices.

The light detecting devices may be fabricated using CMOS compatible processes. (See, e.g., U.S. Pat. No. 7,786,421 entitled “Solid-State Curved Focal Plane Arrays” and filed Sep. 13, 2004 and U.S. Pat. No. 8,828,852 entitled “Delta-Doping at Wafer Level for High Throughput, High Yield Fabrication of Silicon Imaging Arrays” and filed Dec. 10, 2010, the disclosures of which are hereby incorporated by reference in their entirety). The light detecting devices may be fabricated from a thin membrane. The thin membrane can be a biocompatible material, for example a silicon or gallium membrane. The membrane can develop a silicon nitride layer or a gallium nitride layer respectively when thinned. The light detecting devices may comprise light detecting device arrays. The light detecting device arrays can be a continuous membrane affixed to the substrates. The light detecting device arrays can be a series of light detecting device wafers disposed in a single layer on the substrates. One side of the thinned membrane is passivated such that it can detect light, for example UV photons. The thinned membrane may be passivated through delta-doping. The thinned membrane is passed through the ultra-high vacuum chamber, with a base pressure less than approximately 2×10⁻¹⁰ Torr, of a molecular beam epitaxy system. The molecular beam epitaxy system allows the growth of an epitaxial layer on one side of the thinned membrane. For example, through electron beam evaporation of elemental silicon and thermal evaporation of elemental boron by the molecular beam epitaxy system a p+ silicon delta-doped layer may be deposited about 0.5 nanometers above the surface of one side of the thinned membrane. An additional capping layer of epitaxial silicon may be grown that may then be exposed to the air to allow for oxidization. The oxidized capping layer can protect the delta-doped layer. The delta-doped thin membrane may then be conformed to a curved shape (concave or convex) or any shape of interest. The curvature of radius can be limited by the strain limit of the membrane material and the size and shape of the detection probe housing and/or detection system camera housing. To achieve a desired radius of curvature for the housing of the detection system, the light detecting devices may comprise light detecting device arrays. The light detecting device arrays can be a continuous membrane affixed to the substrates. The light detecting device arrays can be individual light detecting device wafers disposed in a single layer on the substrates. The light detecting device may either be front-lit or back-lit. A light detecting device may be back-lit when the light emitted from the excited biomarkers and/or bacteria passes through the back of the light detecting device. A light detecting device may be front-lit when the light detecting device is configured so the light emitted from the biomarkers passes through the front of the light detecting device.

The anti-reflective coatings for the light detecting devices may be fabricated (See, e.g., U.S. Pat. No. 8,697,474 entitled “Methods to Fabricate and Improve Stand-Alone and Integrated Filters” and filed Jan. 13, 2011, the disclosure of which is hereby incorporated by reference in its entirety for all purposes.) The wavelength of emitted light the light detecting device can detect may be tailored through the application of an anti-reflective coating (AR coating). The AR coating comprises a thin film of metals, oxide, fluorides, nitrides, carbides, and any combination thereof. The wavelength the light detecting device may be able to detect can depend on the AR coating film material, the thickness of the AR coating film layer, the arrangement of the different AR coating layers, and any combination thereof. The AR coating layers may be deposited through atomic layer deposition (ALD). ALD can occur through the reaction of two or more gaseous chemicals with the surface of the light detecting device. Each reaction can deposit a layer on the surface of the light detecting device. In many embodiments repeated reactions may control the thickness of the different AR coating layers. In some embodiments repeated reactions of different gaseous chemicals can determine the arrangement of the AR coating layers. In several embodiments the light detecting device is comprised of a series of wafer light detecting device, each light detector can be tailored to detect specific wavelengths through individual ALD AR coating configurations. The delta-doped and/or AR coated thinned membrane of the light detecting device may be affixed to a curved substrate, such as a spherical or cylindrical detection system probe housing and/or detection system housing. The detection system probe housing may have multiple configurations to optimize the body cavity being investigated.

### Doctrine of Equivalents

As can be inferred from the above discussion, the above-mentioned concepts can be implemented in a variety of arrangements in accordance with embodiments of the invention. Accordingly, although the present invention has been described in certain specific aspects, many additional modifications and variations would be apparent to those skilled in the art. It is therefore to be understood that the present invention may be practiced otherwise than specifically described. Thus, embodiments of the present invention should be considered in all respects as illustrative and not restrictive.

As used herein, the singular terms “a,” “an,” and “the” may include plural referents unless the context clearly dictates otherwise. Reference to an object in the singular is not intended to mean “one and only one” unless explicitly so stated, but rather “one or more.”

As used herein, the terms “approximately,” and “about” are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

What is claimed is:

1. A biomarker detection system comprising:
at least one light emitting diode, wherein the at least one light emitting diode emits an excitation light that excites a biomarker such that the biomarker auto-fluoresces and emits a light in a wavelength from 100 nm to 450 nm;
at least one light detecting device positioned adjacent the at least one light emitting diode, wherein the at least one light detecting device detects the emitted light and generates a signal; and
at least one signal processor, wherein the at least one signal processor processes the signal to generate a detection result of the biomarker.

2. The detection system of claim 1, wherein the at least one light detecting device comprises a photodiode, a photodiode array, a photodetector, a photodetector array, a CMOS camera, a CCD camera, or a combination thereof.

3. The detection system of claim 1, wherein the at least one light emitting diode is on a curved substrate.

4. The detection system of claim 1, wherein the at least one light detecting device comprises an anti-reflective coating that is selectively transmissible to the emitted light of the biomarker.

5. The detection system of claim 1, further comprising a power source selected from the group consisting of: an AC power supply, a DC power supply, a battery, and a combination thereof.

6. The detection system of claim 5, further comprising a handle, wherein the power source and the at least one signal processor are housed in the handle.

7. The detection system of claim 1, further comprising a signal transmitter selected from the group consisting of: a USB cable, a Bluetooth transmitter, a wireless transmitter, and a combination thereof, wherein the signal transmitter transmits the detection result to a at least one display.

8. The detection system of claim 6, wherein the at least one display is a part of portable device selected from the group consisting of a phone, a tablet, and a laptop computer.

9. The detection system of claim 1, wherein the detection system is configured to insert into a body cavity.

10. The detection system of claim 1, wherein the detection system is configured to survey a surface area.

11. The detection system of claim 1, wherein the at least one detection device is positioned on top of the at least one light emitting diode, wherein the at least one detection device is transmissible to the excitation light.

12. The detection system of claim 1, wherein the at least one detection device and the at least one light emitting diode are positioned in alternating layers.

13. A biomarker detection system comprising:
at least one optical element on a convex surface of a curved substrate;
at least one light emitting diode positioned on a circumference of the at least one optical element, wherein the at least one light emitting diode emits an excitation light that excites a biomarker such that the biomarker auto-fluoresces and emits a light in a wavelength from 100 nm to 450 nm;
at least one light detecting device positioned on a concave surface of the curved substrate, wherein the at least one optical element focuses the emitted light to the at least one light detecting device and the at least one light detecting device detects the emitted light and generates a signal; and
at least one signal processor, wherein the at least one signal processor processes the signal to generate a detection result of the biomarker.

14. The detection system of claim 13, wherein the at least one light detecting device comprises a photodiode, a photodiode array, a photodetector, a photodetector array, a CMOS camera, a CCD camera, or a combination thereof.

15. The detection system of claim 13, wherein the at least one light detecting device comprises an anti-reflective coating that is selectively transmissible to the emitted light.

16. The detection system of claim 13, further comprising a power source selected from the group consisting of: an AC power supply, a DC power supply, a battery, and a combination thereof.

17. The detection system of claim 16, further comprising a handle, wherein the power source and the at least one signal processor are housed in the handle.

18. The detection system of claim 13, further comprising a signal transmitter selected from the group consisting of: a USB cable, a Bluetooth transmitter, a wireless transmitter, and a combination thereof, wherein the signal transmitter transmits the detection result to a at least one display.

19. The detection system of claim 18, wherein the at least one display is a part of portable device selected from the group consisting of a phone, a tablet, and a laptop computer.

20. The detection system of claim 13, wherein the detection system is configured to insert into a body cavity.

21. The detection system of claim 13, wherein the detection system is configured to survey a surface area.

22. A method for detecting biomarkers comprising:
exciting at least one biomarker with at least one light emitting device, wherein the biomarker auto-fluoresces and emits a light in a wavelength from 100 nm to 450 nm;
capturing the emitted light with at least one light detecting device;
processing a signal from the emitted light; and
outputting a detection result of the at least one biomarker.

23. The method of claim 22, wherein the at least one light emitting device comprises a light emitting diode, a laser, and any combination thereof.

24. The method of claim 22, wherein the at least one light detecting device comprises a photodiode, a photodiode array, a photodetector, a photodetector array, a CMOS camera, a CCD camera, and any combination thereof. The method of claim 22, wherein the at least one light detecting device comprises an anti-reflective coating that is selectively transmissible to the emitted light of the biomarker.

26. The method of claim 22, wherein the at least one biomarker is in a body cavity.

27. The method of claim 22, wherein the at least one biomarker in on a surface.
